(19) United States
(12) Patent Application Publication
(10) Pub. No.: US 2022/0264850 A1
(43) Pub. Date: Aug. 25, 2022

(54) DEVICE FOR THE REAL-TIME MONITORING OF LIVESTOCK

(71) Applicant: LIVESTOCK TECHNOLOGY, Clemont Ferrand (FR)

(72) Inventors: Thierry CLAIREAU, Bangkok (TH); Etienne GUEROU, Clermont Ferrand (FR)

(21) Appl. No.: 17/678,489

(22) Filed: Feb. 23, 2022

(30) Foreign Application Priority Data
Feb. 23, 2021 (FR) 2101742

(51) Int. Cl. A01K 29/00; H04Q 9/00

(52) U.S. Cl. CPC A01K 29/00; H04Q 9/00; H04B 1/69

(57) ABSTRACT

A device for the real-time monitoring of livestock includes at least one data collection component equipped with at least one sensor for physiological or physicochemical data and a means for communicating and transmitting data collected by said sensor to at least one fixed component for transmitting and receiving the data connected to at least one remote module for processing the data collected and a control module. Each data collection component is fitted, in a non-removable manner, with a unique identification RFID (Radio Frequency IDentification) data storage element, with at least one sensor of body temperature as physiological data, and with at least one means of communication by UWB (Ultra Wide Band). The fixed components may further include a means of three-dimensional positioning of the data collection component in a delimited space, such that each fixed transmission component is provided with at least one ambient temperature sensor.

This invention relates to a device for the real-time monitoring of livestock.

The term “livestock” includes mammals such as cattle, sheep, goats, horses, pigs, but also other animals such as birds or poultry, fish, or shellfish. The term “livestock farming” refers to the farming of animals on land or at sea, in buildings or livestock spaces, enclosed or in the open air, in aquaculture or shellfish farms, regardless of the type of production involved, for example for meat, milk, eggs, breeding, or for other purposes. Furthermore, they may be either domestic or wild animals, farming being the fact that humans manage, for their own benefit, the life of one or more animals.

### DESCRIPTION OF THE RELATED ART

Due to the size of certain livestock structures which bring together several hundred or several thousand animals depending on the species concerned, this involves constant health and economic monitoring in order to optimize production while preserving animal and human health. It is therefore necessary to collect numerous parameters for each animal raised, these parameters varying according to the species and/or the type of production concerned. As non-limiting examples, parameters may include the identification and/or the pedigree of the animal, its location in the farming structure, physiological parameters relating to the animal such as the temperature, the heat period, the weight of the animal or another parameter. To collect these parameters, various devices are known that use electronic means for collecting, storing, and transmitting data. These means also allow an automatic use, following the identification of the animal, of machines such as automatic concentrate dispensers (ACD), automatic milk dispensers (AMD), automatic weighing machines, milking robots, or milk meters. These means are generally used in cattle farms for dairy production.

Among these electronic means, a method from WO-A-2012/125266 for monitoring cattle in the case of a dairy farm is known. Means of identification are positioned in a stable and make it possible to track animals equipped with RFID tags. The positioning of the animals associated with algorithms makes it possible to track the consumption of water and food by modeling the movements near the water and food sources. Similarly, based on the animals' movements, an estrus index and a health index may be defined. US-A-2018/325382 relates to an ear tag for cattle provided with a body temperature sensor and an ambient temperature sensor. The data is collected and transmitted to a management center. Also, a position sensor of a bovine placed in the identification loop of the animal is known from US-A-2020/0296936. Depending on the position of the animal, a movement of the animal towards a given zone is induced. US-A-2018/0303063 discloses a solution for tracking the animal with a GPS beacon installed in the animal's identification loop. US-A-2019130728 describes a solution for collecting data which, via an RFID tag, are transmitted to terminals placed at various locations on the farm, the owner having access to the data via cloud computing. Other devices, such as that described by WO-A-2020129056, ensure the detection of the animal at a given location and open or close doors to manage the movement of the animal. WO-A-2020185255 uses Bluetooth and Wi-Fi to check if the animal is in a specific area assigned to it. AU-A-2020204591 discloses a capsule swallowed by a ruminant which allows physiological data relating to rumination to be collected and transmitted. EP-B-3071023 relates to a collar incorporating a UWB radar allowing information on the physiological parameters of the animal to be collected, the device comprising separate transmission and reception means. A system marketed under the name SMARTBOW® by the company ZOETIS for monitoring dairy cows is also known. This system uses sensors introduced into the animal's identification loop and collects ear movements characteristic of rumination, heat, as well as the positioning of the cow, in order to issue alerts relating to the state of the animal from the data collected by terminals installed in the stables. The AVERY DENNISON company also markets, under the name SMARTRAC, an RFID tag implanted in the identification tag or on the animal and which provides all the identification and origin information of the animal, including the data relating to its medical treatment.

There are therefore many solutions based on electronics and information technology allowing owners to track their animals and optimize the management thereof with a minimum of movements of the animal and/or the owner. Although such solutions may be used for many species and regardless of the number of animals concerned, the fact remains that the information collected and processed is incomplete and/or specific to a species. The existing devices are generally suitable for outdoor use and not for closed and enclosed spaces such as livestock buildings. In addition, for farming certain species, for example pigs or poultry, with several thousand individuals grouped together in a limited space, even if it is not enclosed, the existing systems have relatively limited performance, both for the type of data collected and for the collection and processing of said data, while often being intended for one animal species and/or for given parameters.

It is this need that the invention proposes to resolve by offering a real-time monitoring device for livestock, which is usable regardless of the number of animals and/or species, suitable for collecting and processing various data in a progressive way, in a closed and enclosed space or at least a delimited, three-dimensional space.

### BRIEF SUMMARY OF THE INVENTION

To this end, the subject of the invention is a device for monitoring livestock in real time comprising at least one data collection component adapted to be fitted on an animal, said component being equipped with at least one physiological or physicochemical data sensor, a means for communicating and transmitting the data collected by said sensor to at least one fixed data transmission and reception component connected to at least one remote module for processing the data collected and to a control module, characterized in that each data collection component is equipped, in a non-removable manner, with a unique identification data storage element of the RFID (Radio Frequency IDentification) type, with at least one sensor for body temperature as physiological data of the animal equipped with the data collection component, at least one means of communication by UWB (Ultra Wide Band) with at least three fixed data transmission and reception components constituting a three-dimensional positioning means of the data collection component in a delimited three-dimensional space and in that each fixed transmission component is provided with at least one sensor of an ambient temperature, as physicochemical data, in the delimited three-dimensional space in which the data collection component is located.

Thus, due to the invention, a means of monitoring animals in real time is provided, which makes it possible to monitor several animals, regardless of their number, their species, and/or the place where they are located, indoors in a livestock building or outside, but in all cases in a delimited three-dimensional space, by integrating both data concerning each animal and data concerning the environment of each animal. The use of positioning in the three dimensions at least by UWB ensures high positioning accuracy of the data collection component, and therefore de facto of the animal when said animal is fitted with the data collection component. In addition, the unique identification data ensures personalized and secure monitoring of each animal. These data are collected, processed, and allow a user to act on the farming conditions and/or on the animal, if necessary, without having to be present at the farming site.

According to advantageous but optional aspects of the invention, such a device may comprise one or more of the following features:

the device comprises at least one so-called active component acting on the animal.

The device comprises at least one so-called active component acting on the three-dimensional space.

The remote data processing module is hosted in the cloud.

The remote data processing module is associated with a learning module.

The control module is associated with a remote control terminal of said module.

The device comprises a reading module communicating with at least one fixed component for transmitting and receiving data.

The reading module communicates with at least one active component acting on the animal or on the three-dimensional space.

### BRIEF DESCRIPTION OF THE SEVERAL VIEWS OF THE DRAWINGS

The invention will be better understood, and other advantages thereof will become clearer, from the following description, which is provided by way of non-limiting example and makes reference to the enclosed drawings, wherein:

FIG. 1 is a stylized side view of an animal, here a pig, equipped with a data collection component according to one embodiment of the invention,

FIG. 2 is a top view, on a larger scale, of the data collection component of FIG. 1 and

FIG. 3 is a schematic view of the use of the device, according to a method in accordance with one embodiment of the invention.

### DETAILED DESCRIPTION OF THE INVENTION

The invention is particularly suitable for raising animals in a delimited, three-dimensional space, in particular a closed and enclosed space, typically a livestock building, it being understood that the invention may also be used outdoors and therefore in an open space, as long as a three-dimensional space is defined. In the case of an enclosed space, it is common for a livestock building to house several hundred or even several thousand animals. Said animals are bred either for their meat or for their milk, their eggs, their fur, etc. Current farms are increasingly streamlined and industrialized, in the sense that production must be achieved with optimal yield, in minimum time, preserving the health and well-being of the animals while respecting the various regulations. The concentration of many animals in a delimited space, which is even more concentrated when it is enclosed, generates constraints to health and the environment, among other things, which require regular monitoring of the animals and daily management of the farming conditions by a staff whose numbers are limited.

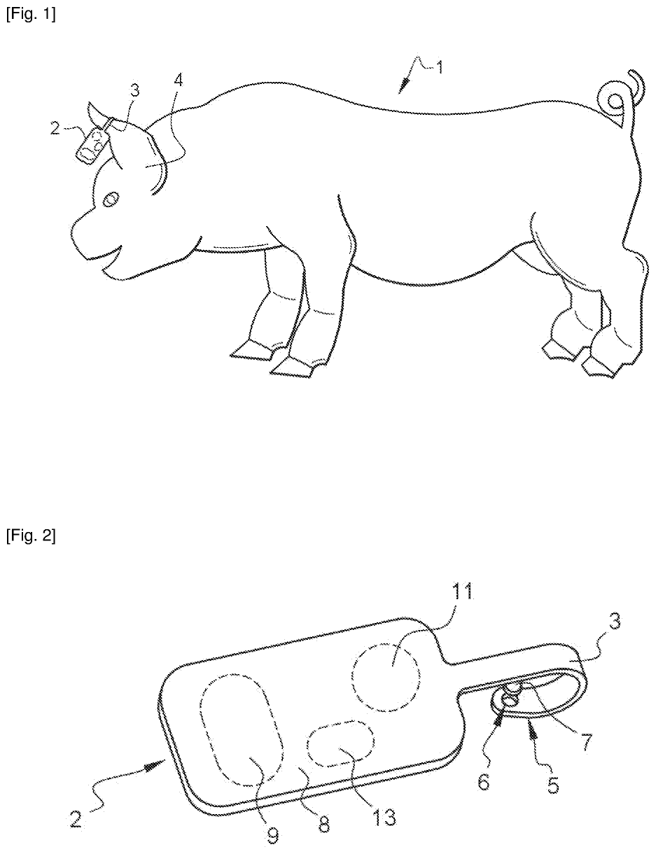

FIG. 1 illustrates an animal 1, in this case a pig, fitted with a data collection component 2, in this case an ear tag. It is easily understood that the invention may be applied to cattle, goats, sheep, horses, poultry, fish, domestic animals or otherwise, and therefore to any animal requiring monitoring in a raising, breeding, or living area, whether this area is open or closed, indoors or outdoors, provided that this area is three-dimensional and delimited. Thus, by way of non-limiting examples, mention may be made of pig, cattle, or poultry farms, horse or sheep farms in intensive or extensive farming or semi-freedom, the animals being grouped together in given places only for certain periods, aquaculture basins at sea or in fresh water, parks or reserves for wild animals, game farming enclosures or, in general, any delimited area in which there is a large number of animals requiring management and monitoring. It is thus conceivable that the device may, as illustrated, be a loop attached to the animal's ear, but it may also be a collar, a ring or a tag attached to the animal's body. In all cases, the shape, weight, dimensions, and/or constituent material of the device are adapted to the animal and its environment.

Here, the loop 2 comprises, in the illustrated embodiment, an attachment lug 3 on an ear 4 of the pig 1. As is apparent from FIG. 2, the attachment takes place, in a manner known per se, by pinching the ear between two constituent parts of the loop 2. By way of illustration, in FIG. 2, a free end 5 of the lug 3 is provided with an orifice 6 for the insertion by force of the free end of a rod 7 fixed to the lug 3. The connection between the rod 7 and the end 5 is permanent, like a rivet, the rod 7 passing through a hole made in the animal's ear. As a known and non-illustrated variant, other modes of attachment are provided, for example, the loop is in two similar parts placed on each side of the ear 4 of the animal 1 or else the lug is rigid, and the orifice is provided on another piece positioned on the other side of the ear 4 of the animal 1. In all cases, the rod is introduced into a hole made in the animal's ear and the attachment of the loop 2 is definitive. It is not possible, accidentally or otherwise, to remove the tag from the animal's ear without destroying the tag or injuring the animal.

The so-called active part 8 of the loop 2 is formed, in the example shown, as a rectangular part made of a rigid material, impervious to environmental conditions and inert with respect to electromagnetic waves. It is made, for example, of thermoplastic polyurethane or TPU.

Typically, the part 8 has sufficient dimensions and thickness, between 30 mm and 100 mm long by 25 mm to 50 mm wide and 3 mm to 18 mm thick, in the case of use on pigs, to allow the insertion into the material of various elements for the identification, positioning, and collection of physiological or physicochemical parameters. In particular, according to the invention, the part 8 comprises a means for communicating and transmitting data or an antenna 9. Such a means uses at least UWB (Ultra Wide Band) technology. It involves wireless technology that uses a wide spectrum of frequencies, namely a bandwidth of at least 250 MHz, or even at least 500 MHz depending on the regulatory body concerned. Here, the bandwidth is between 3.1 GHz and 10 GHz. UWB technology has low power consumption and high reliability. Besides being used for data transmission, UWB is used for positioning and tracking objects in real time. UWB positioning systems offer an accuracy of at least 30 cm or even in some cases between 5 cm and 10 cm, both inside a building and outside, and in two or three dimensions. It is thus possible to know and track the position of the loop 2, and therefore de facto the animal 1 equipped with the loop 2, in a delimited space, enclosed or otherwise, in relation to a surface, for example the ground of a livestock building but also in relation to the volume of the space, thus informing if the loop is on the ground or high up in the livestock building. Such a possibility is suitable for livestock buildings in which farming takes place on several levels. Furthermore, transmission by UWB is not very or not at all sensitive to obstacles. As a result, the constituent materials of the building or present in the livestock space have little or no effect on the transmission of data and communications, and therefore the positioning of the loop 2. In another embodiment, the means for communicating and transmitting data use, in addition to UWB technology, BLE (Bluetooth Low Emission) technology with frequencies between 2.4 GHz and 2.483 GHz. BLE technology offers low power consumption as well as ease of implementation. In addition, the use of Bluetooth allows the use of smartphones as a reading means. Nevertheless, the positioning accuracy is much lower than that obtained by UWB, which limits its use to configurations where the precise position of the animals is not necessary and/or the movement dynamics of the animals are not taken into account. In addition, NFC technology may also be associated for so-called close identification with a reader placed a few centimeters from the loop. This technology is used as an interface to reprogram certain elements of the loop and/or to recharge energy storage components, typically batteries implanted in the loop.

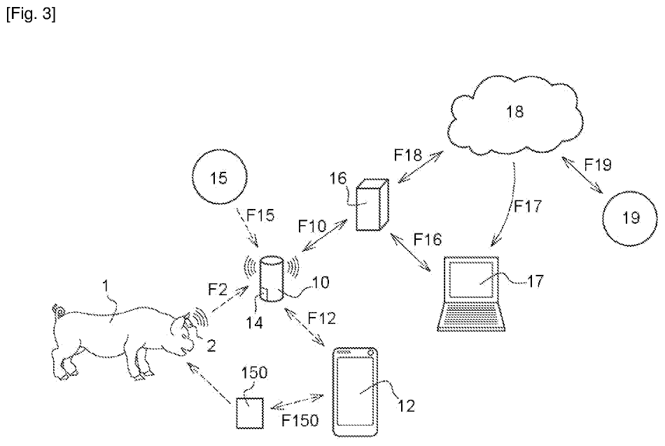

To perform the positioning of the means 9, in a delimited space, enclosed or otherwise, the position of the means 9 is defined with respect to a known and fixed geographical reference. In this case, for positioning in three dimensions, according to the invention, at least three fixed reference points are required. Here the fixed term must be understood as the absence of movement of the reference point during the measurement. Once the measurement or the series of measurements has been made, it is possible to move the reference points to another location, for example another livestock building when the animals are moved to a new building. Alternatively, the points are permanently present in each of the spaces which are or will be occupied by the animals. These reference points, also called terminals, anchors, or beacons, evaluate the distance and the direction of the means 9 with respect to said reference points by measuring the propagation time of the signals, which allows a calculation of the position of the means 9 by trilateration. A reference point is advantageously integrated into a terminal 10, illustrated in FIG. 3. Advantageously, the terminals 10 are positioned in a place where the animals cannot have direct contact with them, in order to avoid any risk of degradation of the terminals by the animals. For this, they are, for example, placed higher than the animals. In another embodiment, a terminal 10 receives data from several means 9, in order to simultaneously manage several components 2. In a variant not shown, the reference point is formed by a box dedicated to or integrated into an object other than terminal 10. In all cases, in the context of the invention, the reference point has a known geographical position. Here, the terminals 10 are made of a rigid material, inert to environmental conditions, resistant to shocks and possible animal bites and inert with respect to the electronic components that it houses. Typically, a terminal 10 is made of PVC (polyvinyl chloride), PU (polyurethane) or acrylic and its dimensions are adapted to the components that it receives and to the space in which it is placed.

The loop 2 also receives an identification data storage element 11. Here the element 11 is a tag of the RFID (radio Frequency IDentification) type. An RFID tag makes it possible to store identification data on an electronic chip associated with an RFID antenna, which allows the exchange of data with a dedicated RFID reader. The reading distance of RFID tags generally varies between one meter and about fifteen meters, depending on the reader used. Here, the reader is either a mobile reader 12 or a fixed reader, advantageously incorporated into the terminal 10. The identification data contained in the RFID tag referenced 11 are unique and cannot be modified. They are therefore specific to the animal 1 wearing the tag 2, provided of course that the tag 2 is attached to the animal 1. In order to ensure the unique and non-reusable nature of the RFID tag, said tag is positioned in a part of the loop which will be destroyed during, for example, the death or the slaughter of the animal. Thus, the RFID tag is placed in the lug 3 of the loop, this lug being cut when the animal is dead or during its slaughter.

The loop 2 also comprises at least one sensor 13 of physiological data relating to the animal 1 fitted with the loop 2. This is a sensor of the body temperature of the animal fitted with the loop 2. Advantageously, it is a temperature sensor in contact with the animal's skin at the attachment zone of the loop 2. It is understood that the loop 2 may comprise other sensors intended to collect other physiological data relating to the animal. This may be, by way of non-limiting examples, heart rate, fat level of the animal measured by the Doppler effect, oxygen level in the blood measured by oximetry, heat detection, the onset of lactation, etc. These sensors placed in the loop 2 may be associated with other sensors or data collection means placed elsewhere on the animal, for example around the neck, the abdomen, or otherwise. In all cases, the sensor 13 and the other sensors are suitable for the data to be measured and collected and also for the animal.

The various elements present in the loop 2, namely the communication means 9, the RFID tag 11, and the sensor(s) 13 are arranged in the mass of the loop 2 in order to be protected from any damage, whether intentional or otherwise, for example when the animal rubs against a hard surface or in the case of a bite, and against environmental conditions. Furthermore, the relative positions of each element 9, 11 and 13 on the loop 2 are defined in order to avoid any interference. In other words, the layout of the elements 9, 11, 13 on the loop 2 is adapted to the numbers and/or types of elements present, as well as to the environment in which the loop 2 will be used. Some of the constituent elements of the loop 2, when they are active elements, for example sensors 13, require energy to operate. Consequently, even if the lifetime of a loop 2 equipped with an energy storage means, for example a battery of the button cell type, is several months, it may be necessary to recharge the loop with electricity. This is done, for example, by induction or by another technique known per se. In another embodiment, a capacitance rechargeable by NFC is used.

The device also comprises at least one sensor of physicochemical data which relates to the three-dimensional space in which the animal 1 develops. In this case, at least one of these sensors is a temperature sensor 14, for example placed on the terminal 10 or else at another point away from the terminal 10 but, in all cases, at a place representative of an ambient temperature of the considered space. Hygrometry, luminosity, detection sensors for gases such as CO2, NH3, NOx, CH4 or other gases or other physicochemical data likely to affect the health or behavior of animals or people near the animals or even the three-dimensional space, may equip one or more terminals or may be placed in dedicated areas, as illustrated by the reference 15 in FIG. 3.

Similarly, the space in which the animals are located may comprise active components on the animals and/or on the environmental conditions present in the space. These components are schematically represented with the reference 150. The expression “active component” denotes, for example, distributors of sanitary or medical treatment, weighing plates, foggers, fans, air extractors, radiators, lamps, means for opening and closing doors, shutters, feed and litter dispensers, automatic cleaning or watering components, milking robots, restrainers, or other components known per se. In all cases, the sensors 15 and/or the active components 150 are adapted to communicate, in transmission and/or in reception, with at least the mobile reader 12. It is understood that this reader 12, for example a tablet, a smartphone, or a dedicated reader, makes it possible to receive and transmit data and, de facto, to generate instructions to the various elements with which it is in communication, namely the sensors 15, the active components 150, or even the elements present in the terminal 10.

The device also comprises at least one central control module 16. Said module is advantageously placed in an area to which the animals do not have easy access. Thus, the module 16 is remote from the three-dimensional space wherein the animals are located, for example in an area dedicated to the administration of one or more farms, this area possibly being several kilometers away from the livestock space. The communication between the terminal 10 and the control module 16 takes place according to the double arrow F10 by a wired or wireless means, for example the 3G/4G/5G, LTU, Cat 5/6 networks.

The control module 16 is also in communication, wired or otherwise, according to the double arrow F16 with a control terminal 17. The terminal 17 is, for example a computer or a tablet. It is understood that the terminal 17 may be located in the same area as the module 16 or be several kilometers away from said module. In addition to communicating with the terminal 17, the module 16 also exchanges data, for storage and archiving purposes, according to the double arrow F18 with the cloud referenced 18 in FIG. 3. For the record, the term “cloud” refers to computer services, for example storage, networking, provision of computer applications, via the Internet network by an Internet network access provider. The terminal 17 may also, according to the arrow F17, recover computer services and/or data stored in the cloud 18.

According to an embodiment illustrated in FIG. 3, the device may also comprise learning modules 19 which, by exchanging data according to the double arrow F19 with the cloud 18, make it possible to enhance the data and the computer services present in the cloud 18. Advantageously, the module 19 implements algorithms from the field of artificial intelligence.

The operation of the device is now described with reference to FIG. 3. The device that is the subject of the invention comprises at least one data collection component 2 attached to an animal 1, at least three reference points or terminals 10 arranged in three places in a delimited space intended for the farming of at least one animal, the geographical coordinates as well as the altitude of these locations being known and invariable at least for the time when the animal is present in the space. The terminals 10 are therefore stationary at least for the time of monitoring the animal in the dedicated space. Each collection component 2, here an ear tag, communicates with each terminal 10, according to the arrow F2, to transmit data, by the communication means 9 present in the loop 2, and thus by UWB and/or BLE technology. In particular, the identification data of the animal contained in the RFID tag 11 as well as at least the body temperature of the animal 1 collected by the sensor 13 are sent to the terminal 2 closest to the animal by UWB and/or BLE, according to the arrow F2. Such a data exchange may be done automatically, for example at known and regular intervals but also on demand, under the action of the mobile reader 12. In other words, depending on the needs, a user of the device collects, on demand, data on one or more animals present in one or more dedicated livestock spaces. In effect, the same reader 12 or smartphone may communicate, according to the double arrow F12, with several sets of terminals 10 or with several remote livestock spaces simultaneously if necessary. Thus, a person may remotely monitor and manage several hundred or even thousands of animals and/or several livestock spaces. The reader 12 may also exchange data with active components 150 present in the livestock space, according to the double arrow F150. These active components will carry out, under the action of the reader 12, actions relating to one or more animals 1 and/or to their environments. These include, for example, food distribution, health or veterinary treatment, opening doors, starting up ventilation, air filtration, misting, lighting, heating, distributing food or water, starting a milking robot, weighing, or any other action affecting the animal and/or its environment in order to optimize its farming conditions and/or its biological parameters. In another embodiment, a livestock space accommodates several animal species or animals of the same species but at different stages of development. By way of example, adult animals and juvenile animals raised together, sheep and goats together, geese and ducks together in the same space may be cited. In such a configuration, each of the given animal species or each stage of development of an animal, for example an adult and a juvenile, is associated with terminals 10 and/or sensors 15 and/or given active components 150 but present in the same space.

In order to optimize its actions, the reader 12 indirectly receives, via the links according to the double arrows F12 and F15, data coming from sensors 15 and passing through the terminal 10. Data collected by the sensor 14 mounted on the terminal 10 is also used. It is understood that the data collected is variable, depending on the types of sensors and/or depending on the type of animal farmed and/or the farming conditions. Nevertheless, the data concerning on the one hand the body temperature of the animal taken by the sensor 13 and on the other hand the ambient temperature in the three-dimensional livestock space taken by the sensor 14 are systematically taken into account by the device, regardless of the other data collected by the various sensors.

The data collected, and therefore at least the body and ambient temperatures, are transmitted by the terminal 10 to the central control module 16, according to the double arrow F10. This module 16, which comprises a calculation means, and therefore specific algorithms for the farming and the animals concerned, on the one hand transmits data, via the link F18, to the cloud 18 for storage purposes and, according to a non-compulsory embodiment, for automatic learning purposes, via a dedicated module 19. It is thus possible to permanently adapt the operation of automatic devices relating the farming conditions to the needs and/or to the behavior of the animals present in the livestock space.

The control terminal 17, which may or may not be remote from the control module 16, allows at least one user to read the data collected and/or stored in the cloud 18, to induce actions via the links F16 and F17 by acting on the control module 16. The user may also plan future actions on the animals, on the farming conditions, or on the collection and/or enhanced value of the farming products. Examples include the distribution of food, medicine, changes in temperature, humidity in the livestock space, triggering of orders for products, materials, the initialization of collection of the product provided by the animals such as milk or eggs or the animals themselves with a view to their slaughter or their movement to another livestock space. Certain actions may be automated, by switching on fans or foggers if a recommended temperature is exceeded in the livestock building.

In all cases, each animal is constantly monitored, both in terms of its biological parameters and its position in the livestock space, due to a precise geolocation by at least three terminals 10. The technique used allows an optimal positioning precision which is less than one meter, along the three axes, regardless of the number of animals present in the space.

The invention makes it possible to monitor and manage, for a single person, continuously and remotely, one or more livestock space, closed or open, housing several hundred animals, with a wide variety of physicochemical or biological parameters, the parameters being easily modifiable. Thus, the device that is the subject of the invention may be implemented in a pig farm, as illustrated, but also in a sheep, goat, cattle, rabbit, poultry, or fish farm. It is also possible to implement it for raising animals in closed but open parks, such as parks for sheep, goats, cattle, horses or for wild animals or game.

1. A device for the real-time monitoring of livestock (1), comprising:
at least one data collection component (2) suitable for fitting on an animal (1), said at least one data collection component being equipped with at least one sensor for physiological (13) or physicochemical data, of a means for communicating and transmitting (9) data collected by said at least one sensor (13) to at least one fixed component (10) for transmitting and receiving data connected to at least one remote module (18) for processing the data collected and to a control module (16),
wherein each data collection component (2) of said at least one data collection component fitted on an animal is equipped, in a non-removable manner, with a storage of unique identification data (11) of a RFID (Radio Frequency IDentification) type, of at least one sensor (13) of body temperature as physiological data of the animal (1) fitted with the data collection component (2), at least one means of communication (9) by UWB (Ultra Wide Band) with at least three fixed components (10) for transmitting and receiving data constituting a means of three-dimensional positioning of the data collection component (2) in a delimited three-dimensional space and in that each fixed component (10) for transmission is provided with at least one sensor (14) of an ambient temperature, as physicochemical data, in the delimited three-dimensional space in which the data collection component (2) is located.

2. The device according to claim 1, further comprising at least one active component (150) acting on the animal (1).

3. The device according to claim 1, further comprising at least one active component acting on the three-dimensional space.

4. The device according to claim 1, wherein the remote data processing module (18) is hosted in a network cloud.

5. The device according to claim 4, wherein the remote data processing module (18) is associated with a learning module (19).

6. The device according to claim 1, wherein the control module (16) is associated with a control terminal (17) remote from said control module.

7. The device according to claim 1, further comprising a reading module (12) communicating with at least one fixed component (10) for transmitting and receiving the data.

8. The device according to claim 7, wherein the reading module (12) communicates with at least one active component (150) acting on the animal or on the three-dimensional space.
